(19) United States
(12) Patent Application Publication
(10) Pub. No.: US 2010/0143535 A1
(43) Pub. Date: Jun. 10, 2010

(54) METHOD FOR PRODUCING COMPOSITION CONTAINING SIALIC ACID COMPOUND

(76) Inventors: Hidemasa Motoshima, Sapporo-shi (JP); Hidetsugu Goto, Sapporo-shi (JP); Nozomi Ueda, Chitose-shi (JP); Yoshiaki Ookubo, Sapporo-shi (JP)

Correspondence Address: FRISHAUF, HOLTZ, GOODMAN & CHICK, PC, 220 Fifth Avenue, 16TH Floor, NEW YORK, NY, 10001-7708, US

(21) Appl. No.: 12/452,452

(22) PCT Filed: Jul. 4, 2008
(86) PCT No.: PCT/JP2008/062152

§ 371 Date: Dec. 30, 2009

(30) Foreign Application Priority Data
Jul. 12, 2007 (JP) 2007-183202

(51) Int. Cl. C12P 19/26; C12G 3/02

(52) U.S. Cl. 426/11; 435/84

(57) ABSTRACT

Provided is a method of efficiently and economically producing a sialic acid compound-containing composition, in which a content rate of a sialic acid compound is remarkably high, from natural raw materials containing a sialic acid compound such as milk, whey, or a processed product thereof. The present invention provides a method of producing a sialic acid compound-containing composition, comprising: using, as a raw material, a liquid raw material containing a sialic acid compound or a liquid in which a solid raw material containing the sialic acid compound is dissolved or suspended in water; culturing, in the raw material, a microorganism which cannot assimilate the sialic acid compound and can assimilate carbohydrates other than the sialic acid compound contained in the raw material; allowing the microorganism to assimilate the carbohydrates other than the sialic acid compound contained in the raw material; and removing the microorganism.

### TECHNICAL FIELD

The present invention relates to a method of efficiently and economically producing a sialic acid compound-containing composition, in which a content rate of a sialic acid compound is remarkably high, from natural raw materials containing a sialic acid compound such as milk, whey, or a processed product thereof.

In more detail, the present invention relates to a method of producing a sialic acid compound-containing composition, in which the content rate of the sialic acid compound in a total solid content is remarkably increased compared with that in a raw material, from the raw material such as milk, whey, or the processed product thereof, by allowing a microorganism which cannot assimilate a sialic acid compound to assimilate carbohydrates other than the target sialic acid compound, separating microbial cells after culturing, and further, removing inconveniently coexisting substances other than the sialic acid compound by electrodialysis or ultrafiltration from a culture medium after removing the microbial cells.

### BACKGROUND ART

Sialic acid is a collective term for a group of carbohydrates discovered as major products released when glycolipid contained in the brain or mucin contained in saliva is hydrolyzed with a weak acid, and includes many molecular species. At present, sialic acid is defined as a collective term for acyl derivatives of neuraminic acid which is an amino nine-monosaccharide acid. Sialic acid is mainly a component in animal tissues, and distribution of sialic acid is in roughly divided into three fractions in a body. A first fraction is present as a component of a sugar chain in glycoproteins, a second fraction is present as a constitutive sugar of oligosaccharides, and a third fraction is present as a constitutive sugar of glycolipids.

In cow milk, sialic acid is present most abundantly in colostrum, and about 560 mg of total sialic acid per kg of the colostrum is observed. The amount of sialic acid is relatively stable from a transitional period of the colostrum (colostrum obtained after a second milking and milking thereafter) to a late-lactation period, and has been reported to be about 150 to 215 mg per kg of cow milk (e.g., see Non-patent Document 1).

A sialic acid-binding protein contained in cow milk includes κ-casein and immunoglobulin. Sialic acid bound to κ-casein accounts for about 30 to 50% of a total sialic acid amount, and the amount of sialic acid is about 160 mg per kg of the colostrum and about 80 mg per kg of mature milk respectively (e.g., see Non-patent Document 1) . Sialic acid contained in κ-casein is located at a C terminal side of the protein, and when the milk is treated with a milk clotting enzyme (chymosin), sialic acid is migrated to a whey fraction as a glycomacropeptide (peptide fragment corresponding to the 106-169 residue of κ-casein) released as a hydrophilic substance. It has been described that the content of sialic acid is 0.16 to 0.2% of a solid content of sweet whey (e.g., see Non-patent Document 2).

Sialic acid is also present as a component of an oligosaccharide in cow milk, and is present abundantly as 3′-sialyllactose and 6′-sialyllactose (which are hereinafter referred to as sialyllactose). The amount of sialic acid present in glycolipid, i.e., ganglioside is extremely small, and scarcely contributes to the total amount of sialic acid in milk.

κ-casein which is the sialic acid-binding glycoprotein and sialyllactose which is an oligosaccharide-bound sialic acid accounts for 60 to 70% of the total sialic acid contained in the cow milk. It has been also described that most of sialic acid present in the cow milk is N-acetylneuraminic acid.

Sialic acid plays a role in terminating the sugar chain in complex carbohydrates, and is involved in adhesion and fixation on a cell surface. Sialic acid is known to have an action as a receptor when influenza viruses and bacteria adhere to cells and a protection action against hydrolytic enzymes. Therefore, there is an extremely great industrial need for sialic acid as a material for new drug for infection prevention agents and therapeutic agents for influenza. Sialyllactose is known to have many functions, and industrial applicability thereof is extremely wide and various, e.g., for infection prevention agents and growth inhibitors for human immunodeficiency viruses, compositions for external use in skin, insulin secretion accelerators, sialomucin secretion accelerators, suppressor for decrease of brain ganglioside, and the like.

Many methods are known as the method of fractionating and purifying the sialic acid compound such as free sialic acid and sialic acid-binding oligosaccharide from natural materials.

As natural sources from which sialic acid is isolated, chicken eggs and swallow's nests are known in addition to the cow milk and the whey. Naturally occurring sialic acid in a free form is scarcely present in the milk, and is present in a form bound to the protein, the oligosaccharide, and the lipid in a minute amount. Therefore, to fractionate and purify sialic acid in the free form, the method of liberating sialic acid by hydrolyzing with acid or the method of using an enzyme (sialidase) which liberates sialic acid has been employed.

However, to isolate and purify liberated sialic acid, it is necessary to efficiently isolate sialic acid from coexisting lactose, proteins, lipids, and ashes. When sialic acid-binding oligosaccharide is isolated and purified, it is likewise necessary to isolate sialic acid from lactose which is abundantly present.

Therefore, when the sialic acid compound is purified from the cow milk or the whey, it takes a long time to isolate sialic acid from coexisting lactose and organic acids, and the efficiency of concentration, fractionation, and purification is reduced to thereby reduce the economical efficiency.

Here, conventional isolation technologies for the sialic acid compound are reviewed in separate cases of free sialic acid and of sialic acid-binding oligosaccharide.

With regard to the method of isolating free sialic acid, a method of preparing sialic acid is described in Patent Document 1, in which a sialic acid-containing hydrolysis solution obtained by hydrolyzing a sialic acid-containing milk substance with acid to liberate sialic acid is desalted by electrodialysis followed by being purified by the electrodialysis again to yield sialic acid. In this method, because free sialic acid is concentrated and purified by the electrodialysis alone, a long time is required, and thus this method is not suitable for mass production in terms of cost. A large amount of a waste solution containing lactose and proteins to which acid has been added is produced. Even if lactose may be produced from this solution, the economic efficiency is poor because a relative price of lactose is low.

A method of purifying sialic acid is disclosed in Patent Document 2, in which sialic acid is obtained by at least two stages of electrodialysis from a hydrolysate of a natural matter containing sialic acid using at least two types of ion exchange membranes which are a loose membrane through which sialic acid can be passed and a tight membrane for desalting. However, a large amount of electric power is consumed by the two stages of the electrodialysis, and thus this method is uneconomical. Further, it is difficult to isolate sialic acid from the coexisting other components. This method is insufficient in terms of economic efficiency and purity of resulting free sialic acid. Also in this case, when sialic acid contained in only a minute amount in the raw material is recovered, a large amount of the waste solution containing lactose and proteins to which acid has been added is produced. Even if lactose may be produced from this solution, the economic efficiency is poor because the relative price of lactose is low.

In addition, many patents for isolating free sialic acid are available, but there is no patent which discloses a method of producing a large amount of sialic acid with high economic efficiency.

Many patents relating to isolation and purification of sialic acid-binding oligosaccharide and sialic acid-binding peptide (glycomacropeptide) are available.

A method of preparing desalted concentrated milk and desalted milk powder containing a sialic acid compound is disclosed in Patent Document 3, in which whole milk, whey, a lactose mother solution, defatted milk, or butter milk containing the sialic acid compound is desalted by combining a cation exchange resin and a strongly basic ion exchange resin, followed by concentration of the obtained permeate or further production of a dry powder from the solution. However, there is only as described in examples of Patent Document 3 that the content rate of sialic acid compound in the solid content can be increased to about 0.5%. Thus, the sialic acid compound prepared by the method in Patent Document 3 is insufficient in terms of purity thereof.

A method of preparing sialic acid-binding oligosaccharide is disclosed in Patent Document 4, in which an ultrafiltration permeate of milk raw material or molasses derived from lactose production process containing a sialic acid-binding oligosaccharide is desalted by the electrodialysis and applied through a column filled with an anion exchange resin to adsorb the sialic acid-binding oligosaccharide to the resin followed by eluting (performing a chromatographic treatment), then pH of a resulting solution is adjusted to near neutral, and a resulting solution is desalted again by the electrodialysis. However, it takes a long time to adsorb the entire raw material to the anion exchange resin and elute the sialic acid-binding oligosaccharide alone. Additionally, an equipment investment for the resin used for the chromatographic treatment is large. Thus, the production efficiency and the economic efficiency are poor in this method.

Further, in Patent Document 5, a method of isolating sialic acid-binding oligosaccharide is disclosed, in which a raw material solution containing the sialic acid-binding oligosaccharide is supplied to a simulated moving bed chromatographic isolation apparatus using a cation exchange resin as an isolating agent to isolate a fraction containing the sialic acid-binding oligosaccharide. However, this method is also based on the chromatographic treatment, and is not practical in terms of production cost.

Of the sialic acid compounds, with regard to the method of producing free sialic acid, it has been known that N-acetylneuraminic acid can be produced by hydrolyzing colominic acid produced by Escherichia coli, in addition to the method of isolating and purifying sialic acid from the milk, the whey, or a defatted egg yolk, for instance. Colominic acid in an optional amount can be produced by culturing (e.g., see Patent Document 6). However, in this method, for isolation from the medium, it is necessary to go through the same purification step as that in the isolation from the milk, and because the microorganism used for the production is Escherichia coli, this is not suitable as a food.

As described above, the naturally occurring sialic acid compound is mainly contained in the materials derived from animals, in particular, abundantly contained in the milk, the whey, and derivations thereof, and often isolated industrially from the whey. Of those, sialic acid-binding oligosaccharide is particularly abundantly contained as sialyllactose in the colostrum, and hence often isolated from the colostrum. Here, the colostrum refers to cow milk secreted by a dairy cow within 5 days after delivering a calf.

The amount of lactose contained in both the colostrum and the whey is much larger than that of the sialic acid compound, and it is necessary to isolate the sialic acid compound from an overwhelmingly large amount of lactose. Therefore, the isolation from lactose is one of large factors to reduce the economic efficiency of the method of purifying the sialic acid compound in any conventional methods of purifying the sialic acid compound including those in the above-mentioned Patent Documents 1 to 5. Further, in the conventional purification methods, for isolating the sialic acid compound from the whey, the whey to which the acid has been added to liberate sialic acid is produced in a large amount. Even if lactose may be produced from the whey solution, an economic value of lactose is low. Thus, after all, a large amount of waste is produced, which has been a large problem.

Non-patent Document 1: Journal of Dairy Science, 84, pp. 995-1000, (2001)

### Non-patent Document 2: Journal of Agricultural and Food Chemistry, 47, pp. 2613-2616, (1996)

### Patent Document 1: JP 07-103139 B

### Patent Document 2: JP 2899844 B

### Patent Document 3: JP 04-69978 B

### Patent Document 4: JP 63-28428 B

### Patent Document 5: JP 3368389 B

### Patent Document 6: JP 2620795 B

### DISCLOSURE OF THE INVENTION

### Problems to be Solved by the Invention

It is an object of the present invention to provide a method of efficiently and economically producing a sialic acid compound-containing composition, in which a content rate of a sialic acid compound is remarkably high, from natural raw materials containing a sialic acid compound such as milk, whey, or a processed product thereof.

### Means for Solving the Problems

As a result of an extensive study to solve the above-mentioned conventional problems, the inventors of the present invention found that a relative concentration of a sialic acid compound based on a total solid content in a solution after culturing can be exponentially increased by culturing a microorganism which cannot assimilate the sialic acid compound, can assimilate major carbohydrates other than the sialic acid compound, and, if possible, further can assimilate organic acids, allowing the microorganism to assimilate coexisting lactose in a large amount, the other carbohydrates, and the organic acids in the presence of milk, whey, or a processed product thereof containing the sialic acid compound such as free sialic acid or a sialic acid-binding oligosaccharide in a minute amount, and removing lactose, the other carbohydrates and the organic acids as a microbial cell form. The inventors of the present invention also found that the subsequent isolation and purification of the sialic acid compound can be easily performed.

Further, it was found that yeast belonging to the genus Kluyveromyces is suitable as the above-mentioned microorganism.

The inventors of the present invention also found that the relative concentration of the sialic acid compound can be further exponentially increased in the sialic acid compound-containing composition by filtrating the sialic acid compound with an ultrafiltration membrane having a molecular cutoff which is slightly larger than a molecular weight of the sialic acid compound, i.e., the ultrafiltration membrane having the molecular cutoff, through which the sialic acid compound can pass, before or after subjecting to the culturing of the microorganism which cannot assimilate the sialic acid compound, and subsequently desalting by electrodialysis.

Further, the inventors of the present invention found that a sialic acid compound-containing composition containing alcohol can be produced by performing alcohol fermentation simultaneously when the microorganism is allowed to assimilate the carbohydrates other than sialic acid compound and the organic acids.

As described above, it was demonstrated that the sialic acid compound-containing composition and valuable resources, such as yeast cells and alcohol can be produced simultaneously, and that the economic efficiency in the production of the sialic acid compound-containing composition is remarkably enhanced.

The present invention has been completed based on such findings.

That is, a first embodiment of the present invention provides a method of producing a sialic acid compound-containing composition, comprising: using, as a raw material, a liquid raw material containing a sialic acid compound or a liquid in which a solid raw material containing the sialic acid compound is dissolved or suspended in water; culturing, in the raw material, a microorganism which cannot assimilate the sialic acid compound and can assimilate carbohydrates other than the sialic acid compound contained in the raw material; allowing the microorganism to assimilate the carbohydrates other than the sialic acid compound contained in the raw material; and removing the microorganism.

A second embodiment of the present invention provides a method of producing a sialic acid compound-containing composition according to the first embodiment, in which the microorganism can assimilate an organic acid in addition to the carbohydrates other than the sialic acid compound.

A third embodiment of the present invention provides a method of producing a sialic acid compound-containing composition according to the first embodiment, in which the raw material is one kind or more selected from the group consisting of milk, whey, a mixture of the milk and the whey, a processed product of the milk, a processed product of the whey, and a processed product of the mixture of the milk and the whey.

A fourth embodiment of the present invention provides a method of producing a sialic acid compound-containing composition according to the first embodiment, in which the microorganism cannot assimilate the sialic acid compound and can assimilate lactose.

A fifth embodiment of the present invention provides a method of producing a sialic acid compound-containing composition according to the first embodiment, in which the microorganism cannot assimilate sialic acid and/or sialyllactose and can assimilate lactose, glucose, and galactose.

A sixth embodiment of the present invention provides a method of producing a sialic acid compound-containing composition according to the fifth embodiment, in which the microorganism is yeast.

A seventh embodiment of the present invention provides a method of producing a sialic acid compound-containing composition according to the sixth embodiment, in which the yeast is yeast belonging to the genus Kluyveromyces.

An eighth embodiment of the present invention provides a method of producing a sialic acid compound-containing composition according to any of the first to seventh embodiments, in which the microorganism is cultured in the raw material under a condition in which the microorganism can conduct alcohol fermentation.

A ninth embodiment of the present invention provides a method of producing a sialic acid compound-containing composition according to the eighth embodiment, in which the microorganism is removed after culturing, and alcohol produced by the alcohol fermentation is subsequently isolated by distillation.

A tenth embodiment of the present invention provides a method of producing an alcohol beverage or a liquor, comprising using the sialic acid compound-containing composition obtained by the method according to the eighth embodiment.

An eleventh embodiment of the present invention provides a method of producing a sialic acid compound-containing composition according to any of the first to seventh embodiments, further comprising: before or after culturing the microorganism in the raw material, ultrafiltrating the raw material or a cultured material with an ultrafiltration membrane having a molecular cutoff which is larger than a molecular weight of the sialic acid compound to pass the sialic acid compound into a side of a permeate; and further desalting the permeate by electrodialysis.

### EFFECT OF THE INVENTION

The present invention can provide the method of efficiently and economically producing a sialic acid compound-containing composition, in which the content rate of the sialic acid compound is remarkably high, from natural raw materials containing a sialic acid compound such as milk, whey, or a processed product thereof.

The sialic acid compound-containing composition provided by the present invention can be utilized as a raw material for producing foods and pharmaceuticals containing the sialic acid compound because the content rate of the sialic acid compound is extremely high.

The present invention is a method involving the culturing using only the safe materials for human beings as a method of remarkably increasing the content rate of the sialic acid compound. According to the present invention, a scale-up is easily made, and hence, the method of the present invention is highly predominant economically compared with the conventional methods. A majority of the solid content in the medium can be recovered as microbial cells after culturing, and utilized as valuable resources such as yeast cells, feeds for domestic animals, raw materials for yeast extracts, and raw materials for enzymes. Thus, a subsequent load of a waste treatment can also be reduced.

Further, according to the present invention, it is possible to provide the method of producing a sialic acid compound-containing composition in which alcohol can be contained simultaneously. Accordingly, alcohol and the sialic acid compound-containing composition can be produced simultaneously. Further, alcohol beverages and liquors containing the sialic acid compound can be produced.

### BEST MODE FOR CARRYING OUT THE INVENTION

The present invention is described in detail below.

The present invention relates to a method of producing a sialic acid compound-containing composition in which a content rate of a sialic acid compound in a solid content is remarkably increased compared with a raw material, by allowing a microorganism which cannot assimilate the sialic acid compound to assimilate carbohydrates other than the sialic acid compound in the natural raw material containing the sialic acid compound such as milk, whey, or a processed product thereof, and isolating microbial cells after culturing.

That is, the present invention is a method of producing a sialic acid compound-containing composition, comprising: using, as a raw material, a liquid raw material containing a sialic acid compound or a liquid in which a solid raw material containing the sialic acid compound is dissolved or suspended in water; culturing, in the raw material, a microorganism which cannot assimilate the sialic acid compound and can assimilate carbohydrates other than the sialic acid compound contained in the raw material; allowing the microorganism to assimilate the carbohydrates other than the sialic acid compound contained in the raw material; and removing the microorganism.

The present invention also relates to a method of producing a sialic acid compound-containing composition in which the content rate of the sialic acid compound is higher, by performing ultrafiltration using an ultrafiltration membrane having a molecular cutoff which is larger than that of the sialic acid compound to pass the sialic acid compound into a side of a permeate, and by electrically dialyzing the sialic acid compound, before or after subjecting the raw material to the culturing.

The “sialic acid compound” in the present invention indicates free sialic acid or a sialic acid-binding carbohydrate which is a compound to which sialic acid has been bound.

Free sialic acid indicates sialic acid which is liberated as a molecule of a single substance. Hereinafter, when describing simply as “sialic acid” is described simply, this term means free sialic acid.

The sialic acid-binding carbohydrate can include specifically sialyllactose which is sialic acid-binding oligosaccharide. Sialyllactose can include specifically 3′-sialyllactose and 6′-sialyllactose.

Sialic acid-containing protein and sialic acid-binding lipid cannot be concentrated and recovered by the production method of the present invention, and thus are not included in the sialic acid compound obtained by the production method of the present invention. In this regard, however, sialic acid molecules included in casein, immunoglobulin, and glycomacropeptide which are the sialic acid-containing glycoproteins can be recovered as free sialic acid by going through the following hydrolysis step.

The raw material containing the sialic acid compound in the present invention can include the milk and the whey, as well as processed products thereof such as milk powders, whey powders, concentrated whey, concentrated mineral whey, crude lactose, and a mother solution after crystallization of lactose upon producing lactose.

In principle, in addition to the milk, natural materials containing sialic acid and the carbohydrate other than sialic acid, such as swallow's nests (Chinese medicine material) and defatted egg yolk produced upon producing egg yolk oil, can be utilized for producing the sialic acid compound-containing composition by utilizing the principle of the present invention.

The milk and the whey used in the present invention are not limited to those milked from the cow, and those milked from the domestic animals such as a goat, a horse, a camel, and a sheep can be used. Here, the cow milk or the cow colostrum is preferable as the milk. Here, the term of the colostrum generally indicates milk secreted within 5 days after parturition of the cow, and is discriminated from ordinary cow milk secreted thereafter. In Japan, they are defined by Ministerial Ordinance Concerning Compositional Standard, etc, for Milk and Milk Products (Ministerial Ordinance No. 52 from Ministry of Health and Welfare in 1951). However, in the present invention, the milk is addressed as a concept including the colostrum.

In the present invention, one kind or more selected from the group consisting of the milk, the whey, a mixture of the milk and the whey, processed products of the milk, processed products of the whey, and processed products of the mixture of the milk and the whey are preferable among them.

In particular, it is preferable to use the whey or the defatted milk which is the processed product. Therefore, when the milk is used as the raw material, it is desirable to use the defatted milk obtained by performing an operation such as centrifugation.

The whey suitable for the present invention can include liquid whey produced upon producing cheese or reconstituted whey obtained by reconstituting whey powder with water.

The method of producing a sialic acid compound-containing composition of the present invention is a method targeting any of the molecular species containing the sialic acid compound, and in particular, a method for the purpose of concentrating and recovering the following substances which are abundantly contained in the milk, the whey, and the processed products thereof: “sialyllactose” which is sialic acid-binding oligosaccharide, the sialic acid-binding proteins such as casein, immunoglobulin, and glycomacropeptide, and “free sialic acid” which is liberated by hydrolyzing sialyllactose with acid.

In the present invention, free sialic acid and sialyllactose can be produced by almost the same equipments and methods except for the presence or absence of the hydrolysis step with the acid.

The sialic acid compound-containing composition in the present invention can be produced by the following method.

As the raw material of the present invention, it is possible to use the liquid raw material containing the aforementioned sialic acid compound.

When the milk or the whey is used as the raw material and the yeast is used as the microorganism, the sugars dissolved therein could be present at concentration at which the yeast can be grown sufficiently, and hence, the intact milk or the intact whey can be used directly, or the milk powder or the whey powder dissolved and reconstituted up to the appropriate concentration can be used as the liquid raw material. For example, those prepared to contain the dissolved sugars in the amount of about less than 40% by weight, more preferably about 0.5 to 25% by weight, and optimally about 1 to 16% by weight can be used as the liquid raw material.

The liquid raw material can also liberate the sialic acid compound as free sialic acid by being hydrolyzed with the acid.

Types of the acids which can be used in this step can include sulfuric acid and hydrochloric acid, and preferably it is desirable to use sulfuric acid.

In order to sufficiently liberate free sialic acid in this step, it is desirable to add the acid to the liquid raw material so that about 0.2 N of the acid is contained and hydrolyze at about 80° C. for about one hour.

In the present invention, when the hydrolysis is performed, the sialic acid compound contained in the liquid raw material is liberated as free sialic acid in the liquid raw material, and the sialic acid compound-containing composition in which free sialic acid is abundantly contained can be obtained by going through the following step.

When the hydrolysis is not performed, particularly when the colostrum is used as the raw material, sialyllactose which is sialic acid-binding oligosaccharide is present in the liquid raw material, and the sialic acid compound-containing composition in which sialyllactose is abundantly contained can be obtained by going through the following step.

Then, inconveniently coexisting macromolecular components such as proteins can be eliminated by ultrafiltrating the liquid raw material.

A common flat membrane type ultrafiltration module, spiral mode ultrafiltration module, or hollow fiber mode ultrafiltration module can be used for the ultrafiltration performed in this step.

An ultrafiltration membrane (UF membrane) which can be used in this step is a membrane having a molecular cutoff which is larger than the molecular weight of the target sialic acid compound, and the molecular cutoff of the membrane is preferably 1,000 to 10,000, more preferably 1,000 to 5,000 and most preferably 1,000 to 3,000.

In particular, when free sialic acid is liberated by the hydrolysis, the content rate of the sialic acid compound based on the total solid content can be further enhanced by using the membrane having a molecular cutoff which is larger than the molecular weight of the target sialic acid compound and is preferably 1,000 to 5,000 and more preferably 1,000 to 3,000.

Here, when the molecular cutoff of the ultrafiltration membrane is smaller than the molecular weight of the target sialic acid compound, this case is not preferable because the sialic acid compound cannot pass through the membrane. When the ultrafiltration membrane having the molecular cutoff which is larger than 10,000 is used, there are merits that a permeation flux becomes fast and a treatment speed becomes fast, whereas there is a demerit that the amount of the coexisting macromolecular components is increased in the permeate and a relative concentration of the sialic acid compound is decreased in the permeate.

In the liquid raw material, the carbohydrates (mainly lactose) other than the sialic acid compound, which inconveniently coexist in the liquid raw material, are removed by allowing the microorganism to assimilate the carbohydrates (mainly lactose) other than the sialic acid compound and then removing the grown microbial cells. Thus, the relative concentration of the sialic acid compound based on the total solid content in the raw material can be remarkably increased. This is a basic principle of the present invention, and is collectively referred to as a sugar assimilation method or an SA method.

That is, in the present invention, the content rate of the sialic acid compound based on the solid content can be remarkably increased by culturing, in the liquid raw material, the microorganism which cannot assimilate the sialic acid compound and can assimilate the carbohydrates other than the sialic acid compound contained in the raw material to cause the microorganism to assimilate the carbohydrates other than the sialic acid compound. Further, the content rate of the sialic acid compound based on the total solid content can be further increased by using a microorganism which can assimilate the carbohydrates other than the sialic acid compound and can additionally assimilate organic acids.

In particular, when lactose is assimilated, it is necessary to assimilate both glucose and galactose forming lactose. When a microorganism which hardly assimilates galactose is used, galactose remains, and thus the relative content rate of the sialic acid compound based on the total solid content is decreased.

Almost every yeast can assimilate glucose, and hence, it is necessary to clearly know whether or not galactose is also assimilated by the yeast.

By producing the sialic acid compound-containing composition by utilizing the SA method, the microbial cells and culture products can be produced simultaneously. Thus the economic efficiency is remarkably enhanced.

In an assimilation step in the present invention, the assimilability of the carbohydrates other than sialic acid compound and the organic acids by the microorganism can be enhanced by preparing a component composition and a pH condition to be suitable for the culturing of the microorganism in the liquid raw material prepared by going through the above-mentioned step.

In this step, the component composition and the pH condition suitable for enhancing assimilability by the microorganism may be the condition suitable for the culturing of the yeast when the yeast is used. For example, in the reconstituted whey, the microorganism can be cultured even without adding an additional nutrient, but in order to efficiently assimilate the contained sugars, it is more preferable to add a nitrogen source such as ammonium sulfate and ammonia.

Specifically, the component composition of the medium suitable for enhancing assimilability by the microorganism in the present invention is the liquid raw material prepared by adding 0 to 20%, preferably about 0.5% ammonium sulfate, 0 to 1%, preferably about 0.2% magnesium sulfate, and 0 to 2%, preferably about 0.4% potassium dihydrogen phosphate, and adjusting pH to 3 to 8, preferably about 5.0 to 5.5 using caustic soda.

It is desirable that the liquid raw material prepared to have the component composition and the pH condition suitable for the culturing of the microorganism be disinfected at low temperature at 60 to 90° C., preferably about 63 to 65° C. for 1 to 60 minutes, preferably about 30 minutes to inhibit the growth of unwanted microorganisms. If necessary, it is also possible to disinfect the liquid raw material under the condition at temperature higher than the above-mentioned temperature.

As the microorganism which can be used in the assimilation step of the present invention, any microorganism can be used as long as the microorganism cannot assimilate the sialic acid compound and can assimilate the carbohydrates other than the sialic acid compound contained in the raw material, but it is desirable to use the microorganism which can additionally assimilate the organic acids other than the sialic acid compound.

In the present invention, the microorganism which “cannot assimilate the sialic acid compound” includes not only the microorganism which cannot assimilate microbiologically the sialic acid compound but also the microorganism which may be equivalent to the microorganism which substantially cannot assimilate the sialic acid compound. Specifically, the microorganisms which assimilate the inconveniently coexisting carbohydrates in the medium for about 4 to 72 hours and preferably for about 24 hours, while scarcely assimilate or weakly assimilate the sialic acid compound are also included.

As examples of the microorganisms, yeast is preferable, yeast belonging to the genus Kluyveromyces and the genus Debaryomyces is more preferable, yeast belonging to the genus Kluyveromyces is still more preferable, yeast of Kluyveromyces marxianus and Kluyveromyces lactis is particularly preferable, and yeast of Kluyveromyces marxianus is most preferable.

In order to facilitate the implementation of the present invention, Kluyveromyces marxianus YRC6040 strain which can be suitably used for the present invention was deposited with an accession number NITE BP-373 to Incorporated Administrative Agency, National Institute of Technology and Evaluation, Patent Microorganism Depositary (2-5-8 Kazusakamatari, Kisarazu-shi, Chiba Prefecture, Japan) on May 29, 2007, but it is not impossible to implement the present invention without using this microbial strain. That is, the yeast strain which cannot assimilate the sialic acid compound can also be used among the yeast strains which can assimilate lactose by searching naturally occurring yeasts or the yeasts stored in public institutions for storing microbial strains.

Lactose-assimilating yeasts are extremely varied and include the genus Brettanomyces, the genus Builera, the genus Candida, the genus Debaryomyces, the genus Kluyveromyces, the genus Lipomyces, the genus Pichia, the genus Rhodotorula, the genus Sporobolomyces, the genus Sterigmatomyces, the genus Trichosporon, the genus Rhodosporidium, the genus Leucosporidium, and the genus Cryptococcus. Of those, those which are also lactose-fermentable include the genus Brettanomyces, the genus Candida, and the genus Kluyveromyces. In particular, a typical example of industrially important lactose-fermentable yeast is Kluyveromyces.

In the present invention, the genus Kluyveromyces refers to the taxonomic position of the yeast thereof. However, the term is a concept which includes many synonyms and asexual generation genus.

At present, according to “The yeasts, a taxonomic study”, edited by C. P. Kurtzman and J. W. Fell published by Elsevier in 1998 (ISBN 0444813128), 15 types of yeasts including K. aestuarii, K. africanus, K. bacillisporus, K. blattae, K. delphensis, K. dobzhanskii, K. lactis, K. lodderae, K. marxianus, K. phaffii, K. polysporus, K. thermotolerans, K. waltii, K. wickerhamii, and K. yarrowii are recognized to be belonging to the genus Kluyveromyces.

Of those, an asexual generation of “K. lactis” is Candida speherica. Assimilation and fermentation ability of the yeast asexual generation are generally the same as those of a sexual generation. Further, many synonyms of K. lactis exist such as Saccharomyces lactis, Zygosaccharomyces lactis, Zygorenospora lactis, Guilliermondella lactis, Dekkeromyces lactis, K. marxianus var. lactis, Zygosaccharomyces casei, Zygosaccharomyces versicolor, Saccharomyces sociasii, Zygofabospora lactis, Mycoderma lactis, Torula sphaerica, Torulopsis sphaerica, Cryptococcus sphaericus, and Candida spherica.

On the other hand, the asexual generation of “K. marxianus” is Candida kefyr. Also in this case, assimilation and fermentation ability of the yeast asexual generation are generally the same as those of the sexual generation. There are 70 types or more titles as synonyms for this yeast. Typical examples include Saccharomyces marxianus, Zygosaccharomyces marxianus, Saccharomyces fragilis, Kluyveromyces bulgaricus, Kluyveromyces bulgaricus. var. bulgaricus, Dekkeromyces fragilis, Guilliermondella fragilis, Dekkeromyces fragilis, Kluyveromyces fragilis, Torulopsis kefyr, Candida kefyr, and Candida pseudotropicalis. All of those synonyms are integrated into the title K. marxianus, which is granted to have a priority. However, industrially important types often use the synonym given at the time of identification as it is. For example, Kluyveromyces fragilis (asexual generation thereof being Candida pseudotropicalis) is most preferably used for the alcohol fermentation of whey, and the title K. fragilis is, at present, often used.

The organism applicable to the present invention is desirable to be cultured by using inoculum of over-night culture grown in such as YM or whey medium, and by inoculating at the rate of about one to 20%, preferably about 10% of volume of main culturing media. By increasing the amount of the microorganism to be inoculated, it is possible to reduce a risk of contamination by other microorganisms upon main culture and perform the assimilation step rapidly.

The microorganism can be cultured under any condition in the assimilation step of the present invention as long as the condition is suitable for culturing the yeast.

In a culturing method, the microorganism can be cultured in a conical flask and a jar fermenter as a test, and the culturing on a large scale can be carried out industrially with the use of culture tanks. A continuous culturing is possible with using a continuous mode fermenter. A culturing scale in this step can be scaled up optionally and easily.

Showing the specific culturing method, for example, using the jar fermenter, a solution of the inoculum is added in the amount of 0.1 to 20%, preferably about 10% based on the liquid raw material to the liquid raw material, and the culturing can be performed at a temperature condition of 10 to 45° C., preferably about 25 to 42° C. for about 4 to 72 hours, preferably about 24 hours with stirring at a rotation frequency of 0 to 1,000 rpm, preferably 500 rpm and aerating with an aeration volume of 0 to 1 vvm, preferably about 0.5 vvm.

When the temperature is lower than 10° C. or higher than 45° C., this case is not preferable because the growth of the yeast belonging to the genus Kluyveromyces becomes poor. In the present invention, it is necessary to culture the microorganism until the carbohydrates other than the sialic acid compound and the organic acids are sufficiently assimilated.

The carbohydrates other than the sialic acid compound and the organic acids can be assimilated by the microorganism by culturing the microorganism in the liquid raw material in accordance with the above-mentioned condition. In particular, when milk, whey, or processed product thereof is used as the raw material, lactose which accounts for the large ratio in the total solid content can be assimilated by the microorganism. In particular, when whey is used, lactose accounting for about 80% in the total solid content can be assimilated.

In the present invention, after the assimilation step, by removing the microorganism from the liquid raw material after culturing, carbohydrates other than sialic acid compound and organic acids in the form of being incorporated in the microorganism can be separated and removed from the liquid raw material.

The microorganism may be removed by any method, and can be removed by centrifugation or filtration. Specifically, the microorganism precipitated can be removed by the centrifugation at 1,000 to 8,000 rpm and preferably 3,000 to 8,000 rpm, for instance.

Therefore, by going through the separation and removal step, a medium can be obtained in which the content rate of the sialic acid compound is remarkably increased based on the total solid content contained in the medium. Here, the “medium” indicates the liquid raw material after removing the microorganism from the culturing after the assimilation step.

The microorganism separated and removed in this separation step can also be recovered and used as microbial cell, feed for domestic animal, and raw material for a fraction production of a functional component derived from the microorganism. When the yeast is used as the microorganism, it can also be used as the raw materials for the fraction production of the functional component, specifically for the raw materials of various valuable resources such as production of yeast cell, production of feed for domestic animal, production of yeast extract, production of vitamins, and production of the enzyme such as lactase. In particular, when the removed microorganism is used as feed for domestic animal, it is unnecessary to remove the proteins before culturing, and a nutrient-rich feed for domestic animal is obtained by culturing the suspension as it is and recovering the inconveniently coexisting proteins together with the microbial cells.

According to examples, in the method of the present invention, when the sialic acid compound-containing composition is produced from 1 kg of the whey powder (dry weight) using the yeast, the amount of the microbial cells separated and recovered in the separation step is about 0.1 kg (dry microbial cells).

For example, when Kluyveromyces marxianus YRC6040 strain is used, about 376 U of lactase can be recovered per g of this freeze-dried yeast cells.

As described above, in the present invention, a majority of the organic matters other than the sialic acid compound-containing composition is assimilated by the yeast, and the yeast cells can be physically separated and recovered by the centrifugation. Thus, the amount of the produced wastes is extremely small, and the environmental load is extremely small.

When the yeast such as Kluyveromyces lactis is used in the present invention, lactase is generally secreted out of the microbial cells. Lactase is thus recovered not from the microbial cells but from the medium after culturing by the ultrafiltration. In this case, by the SA method of the present invention, the sialic acid, compound-containing composition, the yeast cells, and lactase can be produced simultaneously. Therefore, it can be said to have the large economic efficiency.

An electrodialysis treatment in the present invention is performed in order to remove inconveniently coexisting low molecular ion species (e.g., sodium ion, calcium ion), and can be performed using an ordinary membrane electrodialyser. As an ion exchange membrane used for the electrodialysis treatment in the present invention, it is desirable to use the membrane having the molecular cutoff of 100 to 300 and preferably about 300. Specifically, Neosepta (manufactured by ASTOM Co., Ltd.) can be used.

Use of the membrane having the molecular cutoff which is larger than the molecular weight of the target sialic acid compound is not preferable because the sialic acid compound flows out to an electrode side.

In the electrodialysis treatment, it is desirable to desalt to decrease an electric conductivity of the medium down to 0.1 to 1.0 ms/cm and preferably about 0.1 ms/cm.

The case of performing the electrodialysis treatment down to only the value of the electric conductivity more than 1.0 ms/cm is not desirable because about 0.05% salts capable of being desalted remain. The case of performing the electrodialysis treatment down to the value of the electric conductivity less than 0.1 ms/cm is not desirable because sialic acid flows out to the electrode side.

As other desalting methods, the methods by gel filtration and reverse osmosis using a nano-filtration membrane are available and applicable.

A “desalted medium” obtained after the desalting step by the electrodialysis treatment can also be clarified by clarification by bentonite, specifically adding Bengel (manufactured by HOJUN Co., Ltd.) to be contained in the amount of about 1%, stirring, then preferably leaving stand overnight, and centrifuging to precipitate and remove the proteins remaining in the desalted medium (clarifying agent treatment step).

A “desalted medium” or a “clarifying agent-treated medium” obtained through the above-mentioned step can be made into a dry matter or dry powder as the sialic acid compound-containing composition in which the content rate of the sialic acid compound in the total solid content contained has been remarkably increased. For example, the dry matter or the dry powder can be made by concentrating by vacuum reduced pressure concentration or reverse osmosis membrane concentration followed by freeze-drying, nitrogen gas flow drying, reduced-pressure drying, or spray drying.

It is possible to dissolve the dry matter or the dry powder made in advance by the technique such as the freeze-drying or the nitrogen gas flow drying into a solvent such as water when it is needed to be used. The sialic acid compound-containing composition obtained as described above can be used for foods, pharmaceuticals, cosmetics, and industrial raw materials, and in addition, can be used for the raw materials for producing purified sialic acid and purified sialyllactose.

The method of producing a sialic acid compound-containing composition of the present invention including the above-mentioned steps is the method in which the cost is low in the production because this method does not include a column chromatography step. The SA method which is a core element of the present invention uses no organic solvent, is based on the culturing of the yeast which has ever been eaten, and thus has the extremely high safety when the product is utilized for the food. Thus, the sialic acid compound made in the present invention is very valuable to be directly utilized as food materials or healthy food materials.

In the present invention, the content rate of the sialic acid compound can be further increased by purifying the medium obtained after the assimilation step and the separation step for the microorganism, the desalted medium obtained after the desalting step, or the clarifying agent-treated medium obtained after the clarifying agent treatment step by anion exchange column chromatography.

In the present invention, the anion column chromatography used for purifying the sialic acid compound indicates a column chromatography in which eluting is performed by using an anion exchange carrier which exhibits a high affinity with the sialic acid compound so that the sialic acid-containing compound is dissociated with high specificity.

As the column filled with the anion exchange carrier which can be used in the present invention, a column in any form can be used as long as the column meets the purpose, and specifically Q Sepharose Fast Flow manufactured by GE Healthcare Bioscience Corp. can be used.

A column having an optional scale can be made with the anion exchange carrier filled therein, and the column can be scaled up if necessary. A plurality of sets of the columns are prepared, so as to perform purification operation simultaneously.

In the anion column chromatography in the present invention, first, the medium, the desalted medium, or the clarifying agent-treated medium is applied and adsorbed to the anion exchange carrier filled in the column.

The sialic acid-containing compound in the components contained in the applied medium or desalted medium is adsorbed to the anion exchange carrier in an acceptable adsorption range of the carrier.

The amount of the medium or the desalted medium depending on a volume of the column can be applied. Specifically, when the column having a diameter of 50 mm and a length of 50 mm is used, 500 mL of the medium or desalted medium can be applied.

Subsequently, inconveniently coexisting substances which have not been adsorbed to the adsorption carrier are removed by sending water to wash the adsorption column. It is desirable to send the water at a flow rate of 5 mL/minute for water sending time of 100 minutes.

After removing the inconveniently coexisting substances which have not been adsorbed, the sialic acid compound can be dissociated, eluted, and recovered from the anion exchange carrier by sending a solution of about 10 mM sodium chloride. The solution is desirably sent at a flow rate of 5 mL/minute for 100 minutes.

The desalted medium eluted from the anion column obtained in the above-mentioned step contains sodium chloride used upon elution. Thus, it is desirable to perform (the second) electrodialysis treatment to desalt again.

The second electrodialysis treatment can be basically performed using the same apparatus and under the same condition as in the first electrodialysis treatment.

The desalted medium eluted from the anion column obtained in the above-mentioned step is the sialic acid compound-containing composition in which the content rate of the sialic acid compound in the total solid content contained has been remarkably increased, and this can be powderized by concentrating by the reverse osmosis membrane or the vacuum reduced pressure followed by the freeze-drying or the spray drying.

The sialic acid compound-containing composition of the present invention can be used as the raw material for any foods. However, specifically, there may be given the healthy foods, beverages, and yogurt, for instance, in which physiological functions of the sialic acid compound are expected. A shape of the food in which the sialic acid compound-containing composition of the present invention can be used as the raw material thereof is not particularly limited. For example, the food can be powdered, pulverized, or granulated, and can be used in a form of being filled in capsules, as well as in a form of solution of being dispersed in water or ethanol and in a form of a tablet obtained by being blended with an excipient.

The powder obtained by drying the desalted medium eluted from the anion column obtained by going through the anion column chromatography step and the second desalting step is the sialic acid compound-containing composition in which the relative content rate of the sialic acid compound in the solid content has been remarkably increased even compared with the liquid raw material as a matter of course, the medium, the desalted medium, and the clarifying agent-treated medium.

In the present invention, in the sialic acid compound-containing composition obtained after the assimilation step and microorganism separation step, the desalting step, the clarifying agent treatment step, and the anion column chromatography step and the second desalting-step, the relative content rate of the sialic acid compound based on the solid content is higher as the step goes on.

The relative content rate of the sialic acid compound based on the solid content in the liquid raw material before performing the assimilation step in the present invention is extremely low, and is 0.19% in the solid content in the case of, for example, the whey.

On the contrary, as for the sialic acid compound-containing composition obtained by the method of the present invention, specifically, the “medium” after the assimilation step and the microorganism separation step contains the sialic acid compound in the amount of 1.13% based on the total solid content. Thus, the content rate of the sialic acid compound can be increased to about 6 times compared with the content rate thereof based on the total solid content in the liquid raw material.

The content rate of the sialic acid compound is 7.07% based on the total solid content in the “desalted medium” obtained after the desalting step through electrodialysis of the medium, and the content rate of the sialic acid compound can be increased to about 37 times compared with the content rate thereof based on the solid content in the liquid raw material.

Further, the content rate of the sialic acid compound is 9.83% based on the total solid content in the “clarifying agent-treated medium” after the clarifying agent treatment step, and the content rate of the sialic acid compound can be increased to about 52 times compared with the content rate thereof based on the solid content in the liquid raw material.

Further, the content rate of the sialic acid compound is 45.1% based on the solid content is contained in the “desalted medium eluted from the anion column” after the anion column chromatography step and the second desalting step, and the content rate of the sialic acid compound can be increased to about 237 times compared with the content rate thereof based on the solid content in the liquid raw material.

The same can be applied to the case of fractionating and purifying sialyllactose as the sialic acid compound from the cow colostrum. For example, the amount of sialyllactose in the solid content in defatted colostrum from the cow is 0.21%. The amount of sialyllactose in the total solid content is 0.66%, which is about 3.1 times that in the defatted colostrum, when this is ultrafiltrated and a permeate after the filtration is used as the medium. The amount of sialyllactose is concentrated to about 8.6 times in the “medium” obtained by applying the SA method of the present invention to subject the above-mentioned medium to culturing and removing the microbial cells. The concentration of sialyllactose is concentrated to about 45 times in the “desalted medium” after electrodialyzing the “medium”.

As for both sialic acid and sialyllactose, the content rate of the sialic acid compound based on the total solid content is enhanced only by applying the SA method of the present invention followed by performing the electrodialysis.

As for a recovery amount of the sialic acid compound-containing composition obtained by the method of the present invention, in the case of producing a dry matter from the desalted medium after the desalting step, for example, when 180 g of the whey powder is used, about 2.8 g of a dry matter of the sialic acid compound-containing composition containing 10.93% sialic acid can be produced, and when 238.2 g of the solid content of the defatted colostrum from the cow is used, about 4.3 g of a dry matter of the sialic acid compound composition containing 9.53% sialyllactose can be produced.

Further, in the case of producing a dry matter from the desalted medium eluted from the anion column after the anion column chromatography step and the second desalting step, for example, when 100 g of the whey powder is used, about 333 mg of a dry matter of the sialic acid compound-containing composition containing 45.1% sialic acid can be produced.

As described above, the sialic acid compound-containing composition in which the content rate of the sialic acid compound is remarkably increased can be produced in the present invention. Further, in the present invention, the sialic acid compound-containing composition containing alcohol can also be produced by carrying out the assimilation step under the condition in which alcohol fermentation is possible.

That is, the sialic acid compound-containing composition containing alcohol can also be produced simultaneously with allowing the microorganism to assimilate the carbohydrates other than the sialic acid compound and the organic acids by culturing the microorganism under the condition suitable for the microorganism to perform alcohol fermentation in the assimilation step.

The component composition of the medium suitable for the alcohol fermentation by the microorganism in the assimilation step may be any component composition suitable for the alcohol fermentation by the yeast.

Specifically, the component composition of the medium suitable for enhancing the assimilability by the microorganism in the present invention is the liquid raw material prepared by adding 0 to 20%, preferably about 0.5% ammonium sulfate, 0 to 1%, preferably about 0.2% magnesium sulfate, and 0 to 2%, preferably about 0.4% potassium dihydrogen phosphate, and adjusting pH to 3 to 8, preferably about 5.0 to 5.5 using caustic soda.

It is desirable that the liquid raw material prepared to have the component composition and the pH condition suitable for the culturing of the microorganism be pasteurized at low temperature of about 63 to 65° C. for about 30 minutes to inhibit the growth of the microorganisms other than the microorganism inoculated in the incubation.

The culturing condition suitable for the alcohol fermentation by the microorganism in the assimilation step of the present invention may be any condition suitable for the alcohol fermentation by the yeast. That is, specifically, the alcohol fermentation can be carried out under the condition of no strong aeration or no stirring.

The microorganism can be cultivated in the conical flask, and the culturing on the large scale can also be performed using the jar fermenters or the culture tanks.

Showing the specific culturing method, using the jar fermenter, the inoculum in the amount of 0.1 to 20%, preferably about 10% based on the liquid raw material can be added to the liquid raw material, and cultured under a temperature condition of 10 to 40° C., preferably about 25° C. for about 24 to 72 hours, preferably about 24 hours with stirring at a rotation frequency of about 0 to 30 rpm, preferably about 30 rpm without performing the aeration.

By culturing the yeast in the liquid raw material in accordance with the above-mentioned condition, alcohol is generated through the alcohol fermentation by the microorganism simultaneously with allowing the microorganism to assimilate the carbohydrates other than the sialic acid compound and the organic acids.

It is also possible to generate alcohol in the amount of 3% or more in the medium in the assimilation step in the present invention.

The carbohydrates other than the sialic acid compound and the organic acids can be separated and removed from the liquid raw material in the form of being incorporated in the microorganism by removing the microorganism after culturing from the liquid raw material after the alcohol fermentation and the assimilation step.

The microorganism may be removed by any method, and specifically, the precipitated microorganism can be removed by the centrifugation at 1,000 to 8,000 rpm and preferably 3,000 rpm.

Therefore, by going through the separation step, the medium containing alcohol in which the content rate of the sialic acid compound based on the solid content has been remarkably increased, i.e., the sialic acid compound-containing composition containing alcohol can be obtained.

The sialic acid compound-containing composition containing alcohol obtained in the above-mentioned step can also be used for the raw material for alcohol beverages and liquors. The alcohol beverages and liquors may be produced by conventional methods, and the alcohol beverages and liquors containing the sialic acid compound can be easily produced by using the sialic acid compound-containing composition containing alcohol obtained in the present invention.

The alcohol beverages and liquors thus produced contain the sialic acid compound derived from the raw material and possess flavors derived from the milk or the whey which is the raw material.

Alcohol contained in the medium obtained in the above-mentioned step can be isolated and recovered by distillation. Alcohol generated in the present invention can be easily isolated from the medium by the distillation, and the sialic acid compound remains in the distillation residue because the sialic acid compound in the medium is not volatilized. In principle, the amount of the sialic acid compound in the solid content in the remaining medium is the same as the amount of the sialic acid compound obtained when the alcohol fermentation is not performed and the microbial cells are removed after the assimilation by the yeast.

The distillation step can be carried out by any ordinary method, and for example, can be carried out using a vacuum distillation can. Spirits as defined by liquor tax law and alcohol for the food and the industry can be produced using alcohol isolated and recovered in the distillation step.

Therefore, in the present invention, alcohol which is the valuable resource, the yeast cells, and the sialic acid compound-containing composition can be produced simultaneously.

The sialic acid compound-containing composition obtained as described above can be contained in various foods. As such a food, specifically, various products such as general foods, healthy foods, various beverages, and yoghurt may be included, for instance.

### EXAMPLES

The present invention is described below in detail in examples or the like, but the scope of the present invention is not limited to these examples or the like.

### Test Example 1

### Screening of Yeast which Cannot Assimilate Sialic Acid and can Assimilate Lactose

A sialic acid assimilability test was performed in a microplate using 42 yeast strains owned by YOTSUBA MILK PRODUCTS CO., LTD. (microbial strains owned by the inventors of the present invention). The microbial strains used were as follows. Hereinafter, YRC represents the strain owned by YOTSUBA MILK PRODUCTS CO., LTD.

1) Yeast Used in Screening

The yeast used in screening were microbial strains, which are preserved by YOTSUBA MILK PRODUCTS CO., LTD., such as Kluyveromyces marxianus YRC6009, Kluyveromyces marxianus YRC6022, Kluyveromyces marxianus YRC6032, Kluyveromyces marxianus YRC6034, Kluyveromyces marxianus YRC6040, Kluyveromyces marxianus YRC6041, Kluyveromyces marxianus YRC6046, Kluyveromyces marxianus YRC6065, Kluyveromyces marxianus YRC6073, K. lactis YRC6050, K. lactis YRC6054, K. lactis YRC6055, K. lactis YRC6056, K. lactis YRC6057, K. lactis YRC6058, K. lactis YRC6059, K. lactis YRC6060, K. lactis YRC6062, K. lactis YRC6066, K. lactis YRC6067, K. lactis YRC6072, K. lactis YRC6080, K. lactis YRC6081, K. wickerhamii YRC6002, K. wickerhamii YRC6017, Debaryomyces hansenii YRC6023, Debaryomyces hansenii YRC6025, Debaryomyces hansenii YRC6026, Debaryomyces hansenii YRC6028, Debaryomyces hansenii YRC6030, Debaryomyces hansenii YRC6077, Debaryomyces hansenii YRC6078, Debaryomyces hansenii YRC6079, Debaryomyces hansenii YRC6084, Debaryomyces hansenii YRC6085, Debaryomyces hansenii YRC6088, Dekkera anomala YRC6014, Dekkera anomala YRC6045, Candida glaebosa YRC6021, Candida humilis YRC6051, Candida humilis YRC6052, and Zygosaccharomyces cidri YRC6037.

The assimilation medium (0.67% yeast nitrogen base, 0.5% carbon sources, pH 5.0) containing the 0.5% carbon sources was sterilized by filtration with the 0.45 μm filter, and dispensed by 20.0 μL in the microplate. The medium containing no carbon source was used as the negative control. One drop of the yeast suspension whose McFarland turbidity was adjusted to 0.5 to 1.0 was inoculated using the Pasteur pipette and cultured statically at 25° C. On the third day and seventh day after starting culturing, the assimilability of the carbohydrates was confirmed by comparing the absorbance at 660 nm as the turbidity of the medium using a microplate reader. When the growth of the microorganism was observed, the microorganism was determined to have assimilability. As the carbon sources, glucose, lactose, galactose, sialic acid, sialyllactose, citric acid, and lactic acid were used.

3) Results

Yeasts preserved by the company and considered to be incapable of assimilating sialic acid but capable of assimilating lactose in accordance with an evaluation by the microplate include K. wickerhamii YRC6002 (1 strain), K. marxianus YRC6009, K. marxianus YRC6022, K. marxianus YRC6032, K. marxianus YRC6034, K. marxianus YRC6040, K. marxianus YRC6041, K. marxianus YRC6046, K. marxianus YRC6065, K. marxianus YRC6073 (a total of 9 strains), K. lactis YRC6056, K. lactis YRC6057, K. lactis YRC6058, K. lactis YRC6072, and K. lactis YRC6080 (a total of 5 strains), and Deb. hansenii YRC6077 (1 strain).

As a result, it was conceivable that there were relatively many strains which cannot assimilate sialic acid in the strains belonging to K. marxianus. The assimilability was further confirmed in the test in test tubes because erroneous determination is potentially given only by the results from the screening test in the microplate.

### Test Example 2

### Confirmation of Assimilability in Culturing in Test Tubes

From the results of the microplate in Test Example 1, a total of 12 strains such as K. wickerhamii YC6002, K. marxianus YRC6009, K. marxianus YRC6022, K. marxianus YRC6032, K. marxianus YRC6034, K. marxianus YRC6040, K. marxianus YRC6041, K. marxianus YRC6046, K. marxianus YRC6065, K. marxianus YRC6073, K. lactis YRC6080, and Deb. hansenii YRC6077, considered to be candidates were submitted to a sialic acid assimilation test for test tube culturing.

A test method is as follows. An assimilation medium (0.67% yeast nitrogen base, 0.5% carbon source, pH 5.0) containing 0.5% carbon source which was composed of glucose, lactose, galactose, sialic acid, and sialyllactose was dispensed by 2 mL in the test tube. A medium in which no carbon source was added was used as a negative control. The medium was sterilized by filtration sterilization with a 0.45 μm filter. One drop of the microbial strain whose McFarland turbidity was adjusted to 0.5 to 1.0 was inoculated using a Pasteur pipette, and cultured at 180 rpm at 25° C. for 2 to 3 days. In order to confirm the growth of the yeast in a whey solution, the microbial strain was likewise inoculated in 5% whey solution (sterilized) and 10% whey solution (sterilized).

A microbial cell turbidity was measured as absorbance at 660 nm in all the media after culturing. In a case where the absorbance exceeded 1 even when the medium was appropriately diluted, the medium was diluted to reduce the absorbance to 1 or lower and the absorbance of the medium was obtained by multiplication by a dilution ratio.

The medium in which the carbon sources were sialic acid and lactose was centrifuged at 3,000 rpm for 10 minutes to remove the microbial cells, and concentrations of sialic acid, sialyllactose, and lactose in each culture supernatant were measured. The concentration of sialic acid was measured by a periodic acid-thiobarbituric acid method (Biochemical Journal, 81: 384-392, 1961). The concentration of sialyllactose was analyzed using an ABOE sugar chain labeling kit (J-Oil Mills Inc.) according to its manual attached to the kit. The concentration of lactose was measured by HPLC equipped with an R1 detector (manufactured by Hitachi, Ltd.) using a normal phase column. The results are shown in Table 1.

The growth was evaluated by (−): not grown (less than 0.1 at OD₆₆₀); (+): grown (less than 5.0 at OD₆₆₀); (++): sufficiently grown (less than 10.0 at OD₆₆₀); and (+++): extremely well grown (over 10.0 at OD₆₆₀). The assimilability was determined by a degree of the growth and contents of sialic acid, sialyllactose, galactose, and lactose.

TABLE 1

| | Growth evaluation of microbial cells | | | | | |
|---|---|---|---|---|---|---|
| | Sialic | | Galac- | | 5% | 10% |
| Test yeast | acid | Sialyllactose | tose | Lactose | Whey | Whey |
| K. wickerhamii | − | − | + | + | ++ | +++ |
| YRC6002 | | | | | | |
| K. marxianus | − | − | + | + | +++ | +++ |
| YRC6009 | | | | | | |
| K. marxianus | − | − | + | + | +++ | +++ |
| YRC6022 | | | | | | |
| K. marxianus | − | − | ++ | ++ | ++ | +++ |
| YRC6032 | | | | | | |
| K. marxianus | + | − | + | + | ++ | +++ |
| YRC6034 | | | | | | |
| K. marxianus | − | − | ++ | ++ | ++ | +++ |
| YRC6040 | | | | | | |
| K. marxianus | − | − | ++ | ++ | ++ | +++ |
| YRC6041 | | | | | | |
| K. marxianus | − | − | ++ | + | +++ | +++ |
| YRC6046 | | | | | | |
| K. marxianus | − | − | ++ | ++ | + | ++ |
| YRC6065 | | | | | | |
| K. marxianus | − | − | ++ | ++ | ++ | +++ |
| YRC6073 | | | | | | |
| K. lactis | − | − | ++ | ++ | ++ | +++ |
| YRC6080 | | | | | | |
| Deb. Hansenii | − | − | ++ | ++ | +++ | +++ |
| YRC6077 | | | | | | |

Any strains shown in Table 1 were scarcely grown by the addition of sialic acid or sialyllactose, and thought to be able to be utilized in the present invention. However, some strains were insufficiently grown in the presence of lactose or galactose. Thus, all of the strains were not necessarily said to be good. Among them, in particular, K. marxianus YRC6032, K. marxianus YRC6040, K. marxianus YRC6041, K. marxianus YRC6046, K. marxianus YRC6073, K. lactis YRC6080, and Deb. hansenii YRC6077 were thought to be able to be used in the present invention. K. wickerhamii YRC6002, K. marxianus YRC6009, K. marxianus YRC6022, and K. marxianus YRC6034 had small assimilability of lactose and galactose, and K. marxianus YRC6065 was grown to the lower extent than the other microbial strains in the whey solution. Thus, these microbial strains were thought to be unsuitable for the present invention.

From the above, K. marxianus YRC6032, K. marxianus YRC6040, K. marxianus YRC6041, K. marxianus YRC6046, K. marxianus YRC6073, K. lactis YRC6080, and Deb. hansenii YRC6077 which could not assimilate sialic acid and sialyllactose and could assimilate glucose, galactose, and lactose were thought to be able to be utilized in the present invention. In these strains, the sialic acid compound added in the medium was not assimilated at all, and the other carbohydrates added were completely assimilated. Therefore, it has been suggested that to implement the present invention, if the yeast strains including those belonging to the genera Kluyveromyces and Debaryomyces are mainly screened by the method shown in this example, the strain required for the present invention can be obtained from a natural world.

When the present invention is implemented, if free sialic acid is recovered from the sialic acid compounds, it is enough not to assimilate sialic acid, or if sialyllactose is recovered, it is enough not to assimilate sialyllactose. In view of the principle of the SA method, even if the sialic acid compound is assimilated slightly, as long as the sialic acid compound is not assimilated during culturing, there is no problem when the present invention is substantially implemented.

Of the above-mentioned yeast strains which can be used in the present invention, the detail tests were performed using K. marxianus YRC6040 having the good results for a growth rate and yield of microbial cell.

### Test Example 3

### Screening of Yeast which Cannot Assimilate Sialic Acid and can Assimilate Lactose

A sialic acid assimilability test was performed in a microplate using 13 yeast strains derived from public institutions for storing microbial strains. The microbial strains used are as follows. Hereinafter, NBRC refers to a strain available from the Biological Resource Center in the Department of Biotechnology of the National Institute of Technology and Evaluation (NBRC), JCM refers to a strain available from the Japan Collection of Microorganisms of the RIKEN BioResource Center, and ATCC refers to a strain available from the American Type Culture Collection.

1) Yeast Used in Screening

The yeast used in screening include K. marxianus NBRC10005 (T), K. marxianus NBRC0260, K. marxianus NBRC0288, K. marxianus NBRC1735, K. marxianus JCM1614, K. marxianus JCM1630, K. marxianus ATCC8554, K. marxianus ATCC10022, K. marxianus ATCC12424, K. marxianus ATCC16045, Deb. hansenii JCM1990 (T), Deb. pseudopolymorphus JCM3652 (T), and Deb. castellii JCM6177 (T). Here, (T) refers to the type strain of the yeast species.

For comparison, K. marxianus YRC6040 selected in Test Examples 1 and 2 was also subjected together with the above-mentioned strains to the test.

2) Method for Assimilability Test Using Microplate

The assimilation medium (0.67% yeast nitrogen base, 0.5% carbon sources, adjusted pH to 5.0 with hydrochloric acid) containing the 0.5% carbon sources was sterilized by filtration with the 0.45 μm filter, and dispensed by 200 μL in the microplate. The medium containing no carbon source was used as the negative control. One drop of the yeast suspension whose McFarland turbidity was adjusted to 0.5 to 1.0 was inoculated using the Pasteur pipette and statically cultured at 25° C. On the third day after starting culturing, the assimilability of the carbohydrates was confirmed by comparing the absorbance at 660 nm as the turbidity of the medium using a microplate reader. When the growth of the microorganism was observed, the microorganism was determined to have assimilability. As the carbon sources, glucose, lactose, galactose, sialic acid, sialyllactose, citric acid, and lactic acid were used. The results are shown in Table 2. In table 2, the evaluation was represented as ∘: grown; Δ: very poorly grown; and x: not grown.

TABLE 2

| | Growth in each carbon source | | | | | | |
|---|---|---|---|---|---|---|---|
| | | | | Sialic | | Citric | Lactic |
| Microbial strain | Glucose | Lactose | Galactose | acid | Sialyllactose | acid | acid |
| K. marxianus | ∘ | Δ | ∘ | x | x | ∘ | ∘ |
| NBRC10005 | | | | | | | |
| K. marxianus | ∘ | ∘ | ∘ | x | x | x | ∘ |
| NBRC0260 | | | | | | | |
| K. marxianus | ∘ | ∘ | ∘ | Δ | Δ | ∘ | ∘ |
| NBRC0288 | | | | | | | |
| K. marxianus | ∘ | ∘ | ∘ | x | x | ∘ | ∘ |
| NBRC1735 | | | | | | | |
| K. marxianus | ∘ | ∘ | ∘ | Δ | Δ | ∘ | ∘ |
| JCM1614 | | | | | | | |
| K. marxianus | ∘ | ∘ | ∘ | Δ | Δ | ∘ | ∘ |
| JCM1630 | | | | | | | |
| K. marxianus | ∘ | ∘ | ∘ | x | x | Δ | ∘ |
| ATCC8554 | | | | | | | |
| K. marxianus | ∘ | ∘ | ∘ | ∘ | x | ∘ | ∘ |
| ATCC10022 | | | | | | | |
| K. marxianus | ∘ | ∘ | ∘ | x | x | ∘ | ∘ |
| ATCC12424 | | | | | | | |
| K. marxianus | ∘ | ∘ | ∘ | x | x | x | ∘ |
| ATCC16045 | | | | | | | |
| Deb. hansenii | ∘ | ∘ | ∘ | Δ | Δ | ∘ | ∘ |
| JCM1990 | | | | | | | |
| Deb. | ∘ | ∘ | ∘ | Δ | x | Δ | ∘ |
| pseudopolymorphus | | | | | | | |
| JCM3652 | | | | | | | |
| Deb. castellii | ∘ | ∘ | ∘ | Δ | ∘ | ∘ | ∘ |
| JCM6177 | | | | | | | |
| K. marxianus | ∘ | ∘ | ∘ | x | x | x | ∘ |
| YRC6040 | | | | | | | |

3) Results

From the results in Table 2, the yeast strains which did not assimilate sialic acid or sialyllactose were widely observed in the yeast strains stored in the public institutions for storing microbial strains, and many yeast strains were thought to be able to be utilized in the present invention.

In particular, of 13 strains screened, K. marxianus NBRC0260, K. marxianus NBRC1735, K. marxianus ATCC8554, K. marxianus ATCC12424, and K. marxianus ATCC16045 may have not assimilated sialic acid and sialyllactose and assimilated the other carbohydrates.

If the SA method of the present invention is applied to the concentration of not sialic acid but sialyllactose, many yeast strains are applicable. That is, at least Deb. pseudopolymorphus JCM3652, K. marxianus NBRC0260, K. marxianus NBRC1735, K. marxianus ATCC8554, K. marxianus ATCC10022, K. marxianus ATCC12424, and K. marxianus ATCC16045 can be used.

Of the above-mentioned strains, the strains which assimilate organic acids such as citric acid and lactic acid are slightly more preferable.

Of those, K. marxianus NBRC0260 was thought to be particularly suitable for the use in the present invention because of the good results for the growth rate and the yield of microbial cell, and thus was used together with K. marxianus YRC6040 strain owned by YOTSUBA MILK PRODUCTS CO., LTD. and selected in the Test Examples 1 and 2 to perform a more precise assimilability test in the flask culturing.

### Test Example 4

### Assimilability Test in Flask Culturing

1) Methods

K. marxianus YRC6040 strain owned by YOTSUBA MILK PRODUCTS CO., LTD. and selected in the Test Examples 1 and 2, and K. marxianus NBRC0260 strain selected in Test Example 3 and derived from the institutions for storing microbial strains were cultured in the flask using the whey as the medium, and the concentrations of lactose, sialic acid and sialyllactose in the culture were measured.

First, 0.2 N sulfuric acid was added to 20% reconstituted whey and the mixture was kept at 80° C. for one hour to liberate sialic acid. Subsequently, the mixture was cooled, neutralized with caustic soda, and then passed through an ultrafiltration membrane having a molecular cutoff of 10,000 (Spiral ultrafiltration cartridge S10Y10 manufactured by Amicon) to remove major proteins. This UF membrane treated 20% whey was diluted with water to prepare a solution containing UF membrane treated 10% whey. At that time, ammonium sulfate, magnesium sulfate and potassium dihydrogen phosphate were added at the final concentrations of 0.5%, 0.2% and 0.4% respectively and pH was adjusted to 5.5. This solution was pasteurized at a low temperature of 63° C. for 30 minutes and used as a medium.

Subsequently, this medium was dispensed by 100 mL in a 500 mL conical flask, and 0.01% sialyllactose was added thereto. After pasteurizing at a low temperature of 65° C. for 30 minutes, an inoculum solution prepared by culturing in the test tube so that the absorbance at 660 nm in YPD medium became 30 to 40 was added to the medium at the concentration of 10%, and cultured at 230 rpm at 25° C.

The concentrations of lactose, sialic acid, and sialyllactose in the medium were measured before culturing and 7 days after culturing. The medium was centrifuged at 3,000 rpm for 10 minutes to remove microbial cells. The concentrations of lactose, sialic acid, and sialyllactose were measured in a culture supernatant.

In this test example, the concentration of lactose was measured by HPLC equipped with the R1 detector (manufactured by Hitachi, Ltd.) using the normal phase column. The concentration of sialic acid was measured by the periodic acid-thiobarbituric acid method (Biochemical Journal, 81: 385-392, 1961). The concentration of sialyllactose was analyzed using an ABOE carbohydrate chain labeling kit (J-Oil Mills Inc.) according to its manual attached to the kit. The results are shown in Table 3.

In order to observe the assimilability of sialic acid, sialyllactose, and lactose simultaneously, sialyllactose was added in this test example.

TABLE 3

| | Days in | Lactose | Sialic acid | Sialyllactose |
|---|---|---|---|---|
| Yeast | culturing (day) | (mg/mL) | (mg/mL) | (mg/mL) |
| NBRC0260 | 0 | 5.19 | 0.25 | 0.10 |
| Id. | 7 | 0.00 | 0.25 | 0.10 |
| YRC6040 | 0 | 5.19 | 0.25 | 0.10 |
| Id. | 7 | 0.00 | 0.25 | 0.10 |

2) Results

As shown in Table 3, even when cultured for such a long period as 7 days under an optimal condition, neither K. marxianus YRC6040 strain nor K. marxianus NBRC0260 strain assimilated sialic acid and sialyllactose at all, and were found to be applicable to the present invention.

From the results in the above-mentioned Test Examples 1 to 4, among 55 strains screened, K. marxianus YRC6040 strain and K. marxianus NBRC0260 strain, K. marxianus NBRC1735, K. marxianus ATCC8554, K. marxianus ATCC12424, K. marxianus ATCC16045, K. marxianus YRC6032, K. marxianus YRC6041, K. marxianus YRC6046, K. marxianus YRC6073, K. lactis YRC6080, and Deb. hansenii YRC6077 selected as the yeast which could assimilate lactose and could not assimilate sialic acid and sialyllactose were concluded to be suitable for the present invention. It was also shown that there were many K. marxianus strains having extremely weak assimilability of sialic acid and sialyllactose though they assimilated sialic acid and sialyllactose slightly.

### Test Example 5

### Microbiological Nature of K. marxianus YRC6040 Strain

The yeast was identified in accordance with a publicly known instructional book. As the representative instructional book which can be used, “The yeasts, a taxonomic study”, edited by C. P. Kurtzman and J. W. Fell published by Elsevier in 1998 (ISBN 0444813128) is available.

1) Nature of YRC6040 Strain

Features and natures of YRC6040 strain are shown below.

YRC6040 strain is the yeast isolated domestically from a traditional milk product. This yeast is grown well on a YM agar medium at 25° C. for 3 days, and forms a cream and butyrous colony having a circular and flat shape (1 to 3 mm)×(1 to 3 mm) having a flat surface and a marginal entire margin. Its microbial cell has a circular or elliptic shape having a size of (about 2 to 5 μm)×(about 5 to 10 μm). This yeast is a budding yeast, can be grown at 37° C. This yeast can assimilate glucose, galactose, lactose, sucrose, and inulin, cannot assimilate maltose, can ferment glucose, galactose, lactose, sucrose, and inulin, and cannot ferment maltose. The homology of a base sequence in a D1/D2 region and a base sequence in an ITS region in 26S rDNA exhibit 100% homology to the regions from known K. marxianus strain.

2) Results

From these data, YRC6040 strain was exactly identified as K. marxianus strain. This microbial strain cannot assimilate both sialic acid and sialyllactose and can assimilate lactose well as shown in Test Examples 1 to 4. This YRC6040 strain was deposited with an accession number NITE BP-373 to Incorporated Administrative Agency, National Institute of Technology and Evaluation, Patent Microorganism Depositary.

### Example 1

### Production of Sialic Acid Compound-Containing Composition from Whey

Example in which a sialic acid compound-containing composition is obtained from whey using K. marxianus YRC6040 strain (NITE BP-373 strain) by the method of the present invention is shown.

1) Methods

First, 0.2 N sulfuric acid was added to 20% reconstituted whey (purification step 1) and the mixture was kept at 80° C. for one hour to liberate sialic acid. Subsequently, the mixture was cooled, neutralized with caustic soda, and then passed through the ultrafiltration membrane having the molecular cutoff of 10,000 (Spiral ultrafiltration cartridge S10Y10 manufactured by Amicon) to remove the major proteins.

This UF membrane treated 20% whey was diluted with water to prepare a solution containing UF membrane treated 10% whey. At that time, ammonium sulfate, magnesium sulfate and potassium dihydrogen phosphate were added at the final concentrations of 0.5%, 0.2% and 0.4% respectively and pH was adjusted to 5.5. This solution was pasteurized at a low temperature of 63° C. for 30 minutes and used as the medium (purification step 2).

Subsequently, K. marxianus YRC6040 strain (NITE BP-373) was cultured in a jar (using a 2 L volume jar fermenter, medium 1.6 L, inoculum 10%, 25° C., 500 rpm, 0.5 vvm, cultured for 24 hours). A medium in which the inoculum was cultured was prepared by culturing in a conical flask so as to satisfy OD₆₆₀ nm=30 to 40 in YPD medium.

After culturing, the medium was centrifuged at 3,000 rpm for 10 minutes using a centrifugal separator to separate the yeast cells and collect the medium (purification step 3). Then, the medium which was a remaining supernatant was desalted to an electric conductivity of about 0.1 ms/cm using an electrodialyser (microacilyzer S3 model manufactured by ASTOM Co., Ltd.) and a membrane AC-220-550 (molecular cutoff 300) (purification step 4).

Bengel (manufactured by HOJUN Co., Ltd.) was added to this desalted medium so as to contain 1% bengel, the mixture was thoroughly stirred, left stand at room temperature overnight, and centrifuged at 3,000 rpm for 10 minutes to precipitate the protein. This is a treatment with a clarifying agent (purification step 5). Subsequently, a supernatant after the treatment with the clarifying agent was freeze-dried to obtain a sialic acid compound-containing composition (purification step 6).

In each purification step shown in Table 4, the weight of the solid content was quantified by a sea sand method and values obtained were shown. The content of lactose was shown by the value quantified by an HPLC analysis using the RI detector. The content of sialic acid was shown by the value quantified by fluorescence-derivatizing each sample with a sialic acid labeling kit manufactured by Takara Bio Inc., and analyzing the sample by HPLC in accordance with the manual attached to the kit. The results are shown in Table 4.

TABLE 4

| | | Total | | Total | Total sialic |
|---|---|---|---|---|---|
| | Total | solid | Total | sialic | acid in solid |
| | weight | content | lactose | acid | content |
| Purification step | (g) | (g) | (g) | (g) | (% by weight) |
| (1) 20% Whey | 900 | 180 | 122.4 | 0.34 | 0.19 |
| solution | | | | | |
| (2) Acid-liberated | 1,600 | 168.5 | 123.2 | 0.34 | 0.2 |
| 10% whey | | | | | |
| solution (medium) | | | | | |
| (3) Medium (after | 1,315 | 29.3 | 0 | 0.33 | 1.13 |
| removal of | | | | | |
| microbial cells | | | | | |
| (4) Desalted | 1,315 | 4.1 | 0 | 0.29 | 7.07 |
| medium | | | | | |
| (5) Medium treated | 1,236 | 3.2 | 0 | 0.31 | 9.83 |
| with clarifying agent | | | | | |
| (6) Freeze-dried | 2.8 | 2.8 | 0 | 0.31 | 10.93 |
| medium | | | | | |

2) Results

As shown in Table 4, lactose was completely assimilated within 24 hours. As a result of performing the membrane electrodialysis, the treatment with the clarifying agent, and the freeze-drying, 2.8 g of the sialic acid compound-containing composition (containing about 11% sialic acid) was obtained. A final recovery rate of sialic acid from the 20% whey solution was 91.2%. In addition, 19.6 g of the yeast cell (dry cell) was also obtained. The dry yeast cell thus obtained can be utilized as a valuable resource for various uses.

### Example 2

### Concentration and Purification of Sialic Acid Using Anion Exchange Column

The medium (500 mL) in the purification step 3 shown in Example 1 was dialyzed to the electric conductivity of about 1 ms/cm using the electrodialyser (microacilyzer S3 model manufactured by ASTOM Co., Ltd.) and the membrane AC-220-550 (molecular cutoff 300) to obtain 500 mL of a desalted medium (dialyzed medium).

Subsequently, 100 mL of this desalted medium was adsorbed to 100 mL of an anion exchange column (Q Sepharose Fast Flow manufactured by GE Healthcare Bioscience Corp.), and eluted using 10 mM sodium chloride to obtain 338 mL of a medium eluted from the anion column.

Subsequently, 300 mL of this medium eluted from the anion column was desalted using the electrodialyser (acilyzer manufactured by ASTOM Co., Ltd.) and the membrane AC-220-550 (molecular cutoff 300) to obtain 308 mL of a desalted medium eluted from the anion column.

This desalted medium (250 mL) eluted from the anion column was freeze-dried to obtain 32.1 mg of a solid matter containing 45.1% sialic acid. The final recovery rate of sialic acid from the 20% whey solution was 77.7%. It was shown that the content of sialic acid in the sialic acid compound-containing composition could be increased by combining the publicly known purification by the anion chromatography with the method of the present invention.

### Example 3

### Production of Sialyllactose-Containing Composition from Cow Colostrum

A representative component of the sialic acid compound in the cow colostrum is sialyllactose. In this example it is shown that a sialyllactose-containing composition can be produced from defatted colostrum obtained by defatting colostrum through centrifugation by almost the same methods as in the production of the sialic acid-containing composition from the whey in Example 1.

1) Methods

Fresh cow colostrum (milk milked within 5 days after the parturition) (purification step 1) was centrifuged to obtain defatted colostrum. This defatted colostrum was passed through the ultrafiltration membrane having the molecular cutoff of 10,000 (Spiral ultrafiltration cartridge S10Y10 manufactured by Amicon) to remove the major proteins.

To the UF membrane treated defatted colostrum, ammonium sulfate, magnesium sulfate and potassium dihydrogen phosphate were added so as to contain 0.5% ammonium sulfate, 0.2% magnesium sulfate and 0.4% potassium dihydrogen phosphate respectively and pH was adjusted to 5.5. This solution was pasteurized at a low temperature of 63° C. for 30 minutes and used as the medium (purification step 2).

Subsequently, K. marxianus YRC6040 strain (NITE BP-373) was cultured in the medium in a jar (using a 2 L volume jar fermenter, medium 1.6 L, inoculum 10%, 25° C., 500 rpm, 0.5 vvm, cultured for 24 hours). A medium in which the inoculum was cultured was prepared by culturing in a conical flask so as to satisfy OD₆₆₀ nm=30 to 40 in YPD medium.

After culturing, the medium was centrifuged at 3,000 rpm for 10 minutes using a centrifugal separator to separate the yeast cells and collect the medium which is a supernatant (purification step 3). Then, the medium thus obtained was desalted to an electric conductivity of about 0.25 ms/cm using an electrodialyser (microacilyzer S3 model manufactured by ASTOM Co., Ltd.) and a membrane AC-220-550 (molecular cutoff 300) (purification step 4).

In each purification step shown in Table 5, the weight of the solid content was quantified by a sea sand method and values obtained were shown. The content of lactose was shown by the value quantified by an HPLC analysis equipped with the R1 detector. The content of sialyllactose was analyzed using an ABOE carbohydrate chain labeling kit (J-Oil Mills Inc.) according to its manual attached to the kit. The results are shown in Table 5.

TABLE 5

| | | | | | Sialyllactose |
|---|---|---|---|---|---|
| | | Total | | | in solid |
| | Total | solid | Total | Total | content |
| Purification | weight | content | lactose | sialyllactose | (% by |
| step | (g) | (g) | (g) | (g) | weight) |
| (1) Colostrum | 1,600 | 238.2 | 65.6 | 0.50 | 0.21 |
| (2) Medium | 1,600 | 80.2 | 54.4 | 0.53 | 0.66 |
| (before | | | | | |
| culturing) | | | | | |
| (3) Medium | 1,380 | 25.5 | 0 | 0.46 | 1.82 |
| (after removal | | | | | |
| of microbial | | | | | |
| cells | | | | | |
| (4) Desalted | 1,380 | 4.3 | 0 | 0.41 | 9.53 |
| medium | | | | | |

2) Results

As shown in Table 5, lactose was completely assimilated within 24 hours after the start of the culturing, and sialyllactose was recovered with the recovery rate of 82%. The content of sialyllactose was concentrated to 9.5% after the membrane electrodialysis. By culturing, 13.9 g of the yeast cell (dry cell) was obtained.

As shown in this example, the powder containing 9.5% sialyllactose can be obtained only by culturing, then removing the microbial cell, and subsequently desalting. Thus, it can be said that this method is the extremely excellent method. The resulting microbial cell can be utilized as the valuable resources, and this method is useful for obtaining the sialic acid compound-containing composition.

The content of sialic acid is also higher in the colostrum than in the whey. Therefore, like the method in Example 2, the defatted colostrum can undergo acid digestion to liberate sialic acid, which can be then utilized as the raw material for producing the composition containing free sialic acid.

### Example 4

### Relationship Between Pore Diameter of Membrane Used Upon Ultrafiltrating Medium and Purity of Sialic Acid Compound

One of conceivable reasons why the content of free sialic acid or sialyllactose in the solid content stays at about 10% in Examples 1 and 3 is that the inconveniently coexisting protein remains in the composition.

Thus, the supernatants after culturing in Examples 1 and 3 were subjected to SDS-PAGE electrophoresis, and it was revealed that the proteins having the molecular weight of about 10,000 were contained.

It is conceivable that such inconveniently coexisting matters can be removed by filtrating the solution after acid digestion the reconstituted whey with the ultrafiltration membrane having the molecular cutoff which is smaller than 10,000.

Thus, the culture supernatant after the electrodialysis treatment shown in Example 1 was ultrafiltrated using the membrane having the molecular cutoff of 3,000, and then the content of sialic acid in the solid content in the permeate thus obtained was increased to about 20%. Therefore, it was found that the molecular cutoff of the ultrafiltration membrane largely affects the content of the sialic acid compound in a final product.

As shown in Example 4, it was revealed that the content of sialic acid in the final sialic acid compound-containing composition was enhanced by purifying the solution, in which sialic acid has been liberated by acid digestion, using the ultrafiltration membrane having the molecular cutoff of 3,000 in advance.

It is thought that this is because a considerable amount of the proteins having the larger molecular weight than the molecular cutoff of the membrane used was able to be removed by using the appropriate ultrafiltration membrane whose molecular cutoff is larger than 309.28 because the molecular weight of free sialic acid is 309.28.

It is predicted that the content of sialyllactose in the sialic acid compound-containing composition can be enhanced by selecting an optimal pore diameter of the membrane with the same idea based on the fact that the molecular weight of sialyllactose is 633.6.

### Example 5

### Production of Sialic Acid Compound-Containing Composition with Alcohol Fermentation

The UF membrane treated 20% whey shown in Example 1 was diluted with water to 10 times to make a UF membrane treated 2% whey. The UF membrane treated defatted colostrum shown in Example 3 was diluted with water to 2 times to make a UF membrane treated 50% defatted colostrum.

To these UF membrane treated 2% whey and UF membrane treated 50% defatted colostrum, ammonium sulfate, magnesium sulfate, and potassium dihydrogen phosphate were added so that 0.5% ammonium sulfate, 0.2% magnesium sulfate, and 0.4% potassium dihydrogen phosphate were attained and pH was adjusted to 5.5. These solutions (each 90 mL) were each placed in a 100 mL volume conical flask, and pasteurized at a low temperature of 63° C. for 30 minutes to use as a medium.

Then, 10 mL of a cultured yeast solution (inoculum) of K. marxianus YRC6040 strain (NITE BP-373 strain) was inoculated to these media, and cultured at 25° C. statically without aeration. The cultured yeast solution (inoculum) was prepared by inoculating K. marxianus YRC6040 strain (NITE BP-373 strain) to YPD medium and culturing at 25° C. for 24 hours with shaking at 180 rpm.

As for the UF membrane treated 2% whey medium, the alcohol concentration, the lactose concentration, and the sialic acid concentration were measured during the culturing. As for the UF membrane treated 50% defatted colostrum medium, the alcohol concentration, the lactose concentration, and the sialyllactose concentration were measured during the culturing. The results are shown in Tables 6 and 7, respectively. When the concentration was measured, the medium was centrifuged at 15,000 rpm for 5 minutes to remove the microbial cells, and the obtained supernatant was subjected to the measurement.

The alcohol concentration was measured using F-Kit Ethanol (manufactured by Boehringer Mannheim). The lactose concentration, the sialic acid concentration, and the sialyllactose concentration were measured by the same method as in Example 1.

TABLE 6

Culturing time

| | 0 hour | 24 hours | 48 hours | 72 hours |
|---|---|---|---|---|
| Lactose (%) | 0.89 | 0.11 | <0.10 | <0.10 |
| Alcohol (%) | 0.00 | 0.38 | 0.44 | 0.46 |
| Sialic acid | 0.03 | 0.03 | 0.03 | 0.03 |
| (mg/mL) | | | | |

TABLE 7

Culturing time

| | 0 hour | 24 hours | 48 hours | 72 hours |
|---|---|---|---|---|
| Lactose (%) | 1.84 | <0.1 | <0.1 | <0.1 |
| Alcohol (%) | 0 | 0.63 | 0.99 | 0.98 |
| Sialyllactose | 0.20 | 0.28 | 0.23 | 0.24 |
| (mg/mL) | | | | |

As shown in Table 6, even when the culturing was carried out in the UF membrane treated 2% whey medium without performing the aeration, it was shown that lactose was almost completely assimilated in 24 hours from the start of the culturing and the decrease of the sialic acid concentration was not observed after the passage of 72 hours. It was also shown that alcohol was generated in the medium.

As shown in Table 7, even when the culturing was carried out in the UF membrane treated 50% defatted colostrum medium without performing the aeration, it was shown that lactose was almost completely assimilated in 24 hours from the start of the culturing and the decrease of the sialyllactose concentration was not observed after the passage of 72 hours. It was also shown that alcohol was generated in the medium.

It was also shown that K. marxianus YRC6040 strain (NITE BP-373 strain) could very rapidly conduct the alcohol fermentation and it was possible to produce the sialic acid compound-containing composition in conjunction with the alcohol production.

From this result, it was shown that according to the method of the present invention, by conducting the alcohol fermentation using K. marxianus YRC6040 strain (NITE BP-373 strain), inconveniently coexisting lactose could be assimilated completely and the sialic acid compound-containing composition, alcohol, and the yeast cells could be produced simultaneously.

### Test Example 6

### Extraction of Lactase from Yeast Cells

The microbial cells of K. marxianus YRC6040 strain (NITE BP-373 strain) obtained in Example 1 were washed with distilled water, and frozen at −80° C. Proteins were extracted from the frozen microbial cells prepared by a freeze-dryer using Y-PER Yeast Protein Extraction Reagent (manufactured by PIERCE). This extraction supernatant was centrifuged at 15,000 rpm for 10 minutes, and its supernatant was used for analyzing lactase.

A lactase activity was obtained by colorimetrically quantifying the amount of ONP (o-nitrophenol) produced by hydrolysis of ONPG (o-nitrophenyl-β-D-galactopyranoside) using Yeast β-galactosidase Assay Kit (manufactured by PIERCE). One unit represents the amount of the enzyme which librates 1 μmol ONP for one minute under the optimal condition.

By the above-mentioned analysis, 376 U of the lactase activity per 1 g of the yeast cells (dry weight) was observed. Lactase derived from the yeast can be purified using means such as ammonium sulfate precipitation and column chromatography. From this fact, it was shown that the yeast cell produced in the present invention can also be utilized for producing the enzyme.

### Example 6

### Production of Sialic Acid Compound-Containing Composition with Alcohol Fermentation

The UF membrane treated 20% whey shown in Example 1 was diluted with water to 2 times to make a UF membrane treated 10% whey.

To this UF membrane treated 10% whey, ammonium sulfate, magnesium sulfate, and potassium dihydrogen phosphate were added so that 0.5% ammonium sulfate, 0.2% magnesium sulfate, and 0.4% potassium dihydrogen phosphate were attained and pH was adjusted to 5.5. These solutions (each 90 mL) were each placed in a 100 mL volume conical flask, and pasteurized at a low temperature of 63° C. for 30 minutes to use as a medium.

Then, 10 mL of a cultured yeast solution (inoculum) of K. marxianus YRC6040 strain (NITE BP-373 strain) was inoculated to these media, and cultured at 25° C. statically without aeration. The cultured yeast solution (inoculum) was prepared by inoculating K. marxianus YRC6040 strain (NITE BP-373 strain) to YPD medium and culturing at 25° C. for 24 hours with shaking at 180 rpm.

As for the UF membrane treated 10% whey medium, the alcohol concentration, the lactose concentration, and the sialic acid concentration were measured during the culturing. The results are shown in Table 8. When the concentration was measured, the medium was centrifuged at 15,000 rpm for 5 minutes to remove the microbial cells, and the obtained supernatant was subjected to the measurement.

The alcohol concentration was measured using F-Kit Ethanol (manufactured by Boehringer Mannheim). The lactose concentration, the sialic acid concentration, and the sialyllactose concentration were measured by the same method as in Example 1.

TABLE 8

Culturing time

| | 0 hour | 48 hours | 96 hours | 168 hours |
|---|---|---|---|---|
| Lactose (%) | 7.02 | 4.97 | 3.18 | <0.10 |
| Alcohol (%) | 0.00 | 0.99 | 1.88 | 3.40 |
| Sialic acid | 0.30 | 0.30 | 0.30 | 0.30 |
| (mg/mL) | | | | |

As shown in Table 8, even when the culturing was carried out in the UF membrane treated 10% whey medium without performing the aeration, it was shown that lactose was almost completely assimilated in 168 hours from the start of the culturing and the decrease of the sialic acid concentration was not observed after the passage of 168 hours. It was also shown that alcohol was generated at the concentration of 3% or more.

From this result, it was shown that an alcohol beverage could be produced using K. marxianus YRC6040 strain (NITE BP-373 strain) according to the method of the present invention. If necessary, inconveniently coexisting lactose can be completely assimilated, and the sialic acid compound-containing composition, alcohol, and the yeast cells can also be produced simultaneously.

### INDUSTRIAL APPLICABILITY

According to the present invention, the sialic acid compound-containing composition in which the content rate of the sialic acid compound is remarkably high can be produced safely, efficiently, and economically from natural raw materials containing a sialic acid compound such as milk, whey, or a processed product thereof. Thus, the sialic acid compound-containing composition can be produced as the raw material for the foods and the pharmaceuticals containing the sialic acid compound. The microbial cells such as yeast cells and culture products can be produced simultaneously and in parallel. Thus, the economic efficiency can be remarkably enhanced.

The sialic acid compound-containing composition can also be utilized as the raw material in non-food fields, e.g., as the raw material for the filters for virus removal and virus filtering agents utilizing a virus binding ability of the sialic acid compound.

Further, according to the present invention, the sialic acid compound-containing composition in which alcohol is contained simultaneously can be produced. Thus, it is possible to produce the alcohol beverages and liquors containing the sialic acid compound.

1. A method of producing a sialic acid compound-containing composition, comprising:
using, as a raw material, a liquid raw material containing a sialic acid compound or a liquid in which a solid raw material containing the sialic acid compound is dissolved or suspended in water;
culturing, in the raw material, a microorganism which cannot assimilate the sialic acid compound and can assimilate carbohydrates other than the sialic acid compound contained in the raw material;
allowing the microorganism to assimilate the carbohydrates other than the sialic acid compound contained in the raw material; and
removing the microorganism.

2. A method of producing a sialic acid compound-containing composition according to claim 1, wherein the microorganism can assimilate an organic acid in addition to the carbohydrates other than the sialic acid compound.

3. A method of producing a sialic acid compound-containing composition according to claim 1, wherein the raw material is one kind or more selected from the group consisting of milk, whey, a mixture of the milk and the whey, a processed product of the milk, a processed product of the whey, and a processed product of the mixture of the milk and the whey.

4. A method of producing a sialic acid compound-containing composition according to claim 1, wherein the microorganism cannot assimilate the sialic acid compound and can assimilate lactose.

5. A method of producing a sialic acid compound-containing composition according to claim 1, wherein the microorganism cannot assimilate sialic acid and/or sialyllactose and can assimilate lactose, glucose, and galactose.

6. A method of producing a sialic acid compound-containing composition according to claim 5, wherein the microorganism is yeast.

7. A method of producing a sialic acid compound-containing composition according to claim 6, wherein the yeast is yeast belonging to the genus Kluyveromyces.

8. A method of producing a sialic acid compound-containing composition according to any of claims 1 to 7, wherein the microorganism is cultured in the raw material under a condition in which the microorganism can conduct alcohol fermentation.

9. A method of producing a sialic acid compound-containing composition according to claim 8, wherein the microorganism is removed after culturing, and alcohol produced by the alcohol fermentation is subsequently isolated by distillation.

10. A method of producing an alcohol beverage or a liquor, comprising using the sialic acid compound-containing composition obtained by the method according to claim 8.

11. A method of producing a sialic acid compound-containing composition according to any of claims 1 to 7, further comprising:
before or after culturing the microorganism in the raw material, ultrafiltrating the raw material or a cultured material with an ultrafiltration membrane having a molecular cutoff which is larger than a molecular weight of the sialic acid compound to pass the sialic acid compound into a side of a permeate; and
further desalting the permeate by electrodialysis.
